(19) United States
(12) Patent Application Publication
(10) Pub. No.: US 2022/0007924 A1
(43) Pub. Date: Jan. 13, 2022

(54) ENDOSCOPE WITH AN ENDOSCOPE SUPPORT COMPRISING A TELESCOPIC ROD AND A BALL JOINT

(71) Applicant: Medautonomic S.R.L., Venezia Mestre (IT)

(72) Inventors: Paolo FABRIS, Thiene, Vicenza (IT); Valerio CIGAINA, Cortina D'Ampezzo, Belluno (IT); Simone CIGAINA, Villorba, Treviso (IT); Arfeo CANAGLIA, Fara Vic., Vicenza (IT)

(73) Assignee: Medautonomic S.R.L., Venezia Mestre (IT)

(21) Appl. No.: 17/288,544

(22) PCT Filed: Oct. 22, 2019
(86) PCT No.: PCT/EP2019/078684

§ 371 Date: Apr. 25, 2021

(30) Foreign Application Priority Data
Oct. 25, 2018 (IT) 102018000009804

(51) Int. Cl. A61B 1/00; F16C 11/06

(52) U.S. Cl. CPC A61B 1/00149; F16C 2316/10; F16C 11/0614

(57) ABSTRACT

An apparatus (1) for exploring an internal cavity of a human or animal body the apparatus comprising an endoscope (2) and a support (3) for the endoscope, whereby said support comprises a flat rest base (4), a telescopic rod (5) having a lower end (6a) and an upper end (6b), a first ball joint (7) connecting said lower end of the telescopic rod to said rest base, an attachment connector (8) disconnectable from said endoscope, and an operative connection means (9) for connecting said connector to said upper end of said telescopic rod.

The present invention relates to an apparatus for exploring an internal cavity of a human or animal body comprising an endoscope and a support for the endoscope.

The scope of application extends, for example, to an apparatus for exploring an internal cavity of a human or animal body comprising a system for implanting any totally implantable device which includes an endoscope and a support for the endoscope.

In particular, the present invention relates to an apparatus for exploring an internal cavity of a human or animal body comprising a gastroscope and a support for precise, adjustable positioning of the gastroscope.

Endoscopy is a medical procedure, both diagnostic and therapeutic, that enables internal organs of a human or animal body to be directly visualised by introducing, though a cavity, a specific instrument: an endoscope.

Endoscopic examinations and operations are performed by medical-surgical operators and can last from a few minutes to several hours.

According to the current endoscopic procedures, the endoscope is manoeuvred and supported directly by the operator, or with the help of nursing staff, for the period of time necessary for carrying out the diagnostic or therapeutic activity.

Above all in the longer, more delicate operations, endoscopic procedures require the operator to support the endoscope in precise positions and/or sequences of positions and manoeuvres. Therefore, at present, the success of examinations and operations without complications for the patient also depend on the degree of experience of the operator in knowing how to support and manoeuvre the endoscope.

The development of new procedural techniques due to the increase, for example, in devices that are totally implantable in the human body, requires a continuous training of operators in order that they may learn to support and manoeuvre the endoscope according to the new procedures.

This implies delays in the possibility of the public benefitting from the most innovative implantation techniques, which, due to limits in the transmission of knowledge, remain restricted to a few specialised facilities.

Moreover, the continuous support of the endoscope by the operator can result in delays and the occurrence of complications for the patient.

The technical task of the invention is to overcome the above-described limits.

The object of the invention consists in providing an apparatus for exploring a cavity of a human or animal body which enables repeatable results to be obtained irrespective of the specific abilities of the operator.

A further object of the invention is to provide an apparatus for exploring a cavity of a human or animal body which reduces the possibility of human errors occurring during endoscopic procedures.

Another object of the invention is to provide an apparatus for exploring a cavity of a human or animal body which facilitates the use of an endoscope during longer, riskier and more delicate operations.

The technical task, as well as these and other objects are achieved, according to the present invention, by providing an apparatus for exploring an internal cavity of a human or animal body comprising an endoscope and a support for the endoscope, characterised in that said support comprises a flat rest base, a telescopic rod having a lower end and an upper end, a first ball joint connecting said lower end of the telescopic rod to said rest base, a disconnectable attachment connector to said endoscope, and an operative connection means connecting said connector to said upper end of said telescopic rod.

The basic idea of the invention consists, therefore, in providing an apparatus for exploring an internal cavity of a human or animal body configured to support and position an endoscope in a precise and adjustable manner with a high versatility of use and manoeuvring ease.

The apparatus is thus capable of supporting and orienting any endoscope in space with very high versatility in adapting to the different positions of use envisaged by different endoscopic procedures.

Said operative connection means preferably comprises an interposing block between the connector and the telescopic rod, a joint connecting the block to the connector and a joint connecting the block to the upper end of the telescopic rod.

Preferably one of the above said joints is a flat joint and the other one is a ball joint.

The apparatus has unlocking means for unlocking the degrees of freedom of the support.

The connector is preferably in the form of a quick coupling.

Said unlocking means preferably comprise pedals.

At least one of said pedals is preferably pivoted on said flat base and at least one of said pedals is pivoted on said telescopic rod.

Said connector and said interposing block are preferably provided with a mutual quick coupling and release means.

In one embodiment, said first ball joint comprises a first ball positioned in a first housing, said first ball comprising a first half-ball and a second half-ball actuable towards the first half-ball in opposition to at least a first spring by a first of said pedals pivoted on said flat base.

In one embodiment, said unlocking means comprise a first mechanism for transforming a rotation of the first pedal into a linear actuation of a first tie-rod for pulling said second half-ball towards said first half-ball.

In one embodiment, said telescopic rod comprises a first tube having said lower end and a second tube having said upper end and which is slidable along said first tube so as to adjust the distance between said upper end and said lower end, said first half-ball extending with a vertical pin for fixing to said flat base.

In one embodiment, said joint connecting said block to said upper end of said telescopic rod is a second ball joint comprising a second ball positioned in a second housing, said second ball comprising a first half-ball and a second half-ball actuable towards the first half-ball of the second ball in opposition to at least a second spring by a second pedal pivoted on said flat base, said unlocking means comprising a second mechanism for transforming a rotation of the second pedal into a linear actuation of a second tie-rod for pulling said second half-ball of said second ball towards said first half-ball of said second ball.

In one embodiment, said first half-ball of said second ball is fixed to said second tube, said second tie-rod is solidly joined in rotation but not in translation to said first tie-rod and said second mechanism comprises inclinable spacer pins for transforming a rotation of the first tie-rod into a rototranslation of the second tie-rod.

Additional features and advantages of the invention will be more apparent from the description of a preferred, but non-exclusive, embodiment of the implantation instrument according to the invention, illustrated by way of non-limiting example in the appended drawings, in which:

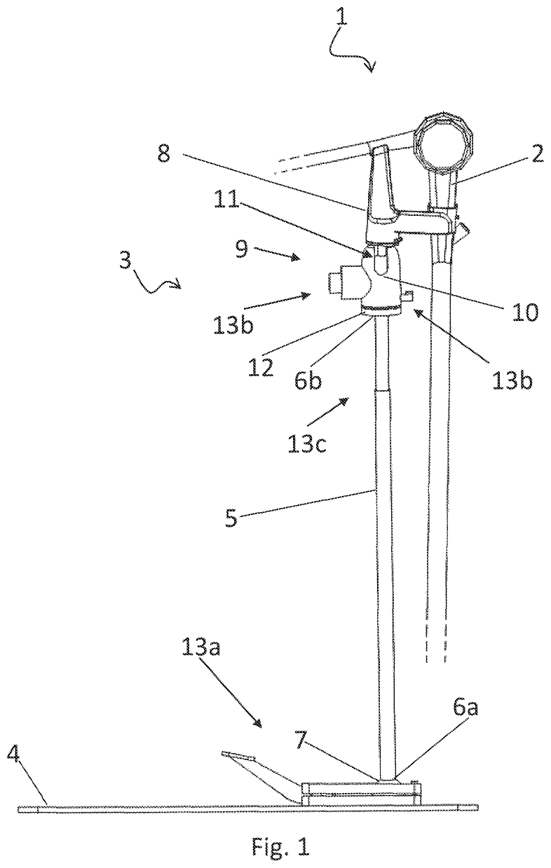

FIG. 1 shows a side view of an apparatus for exploring an internal cavity of a human or animal body according to a first embodiment of the present invention;

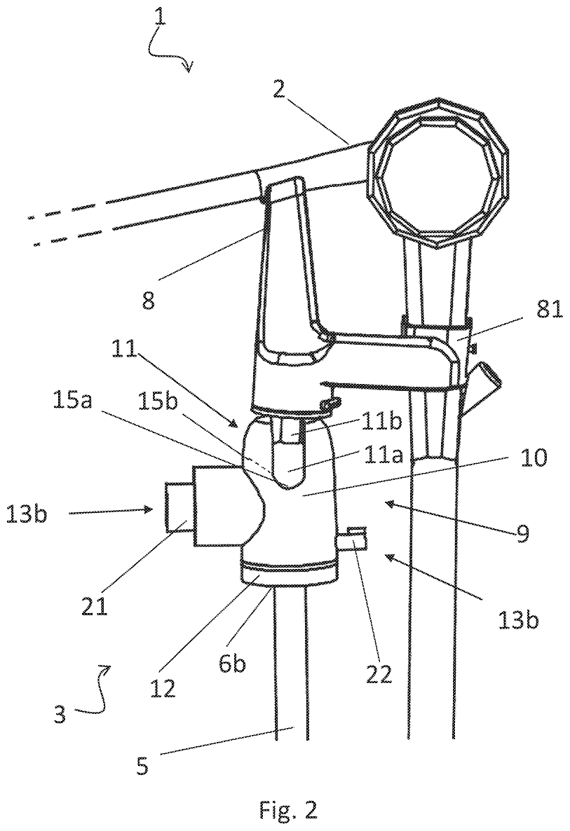

FIG. 2 shows a detailed side view of the operative connection means and of the connector of the apparatus of FIG. 1;

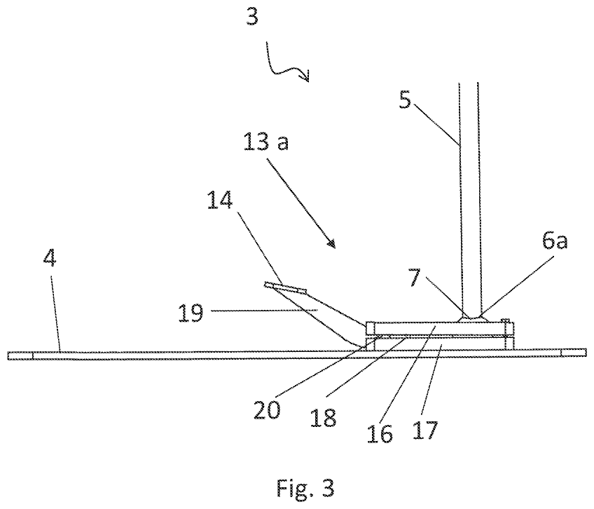

FIG. 3 shows a detailed side view of the rest base and of the ball joint of the apparatus of FIG. 1;

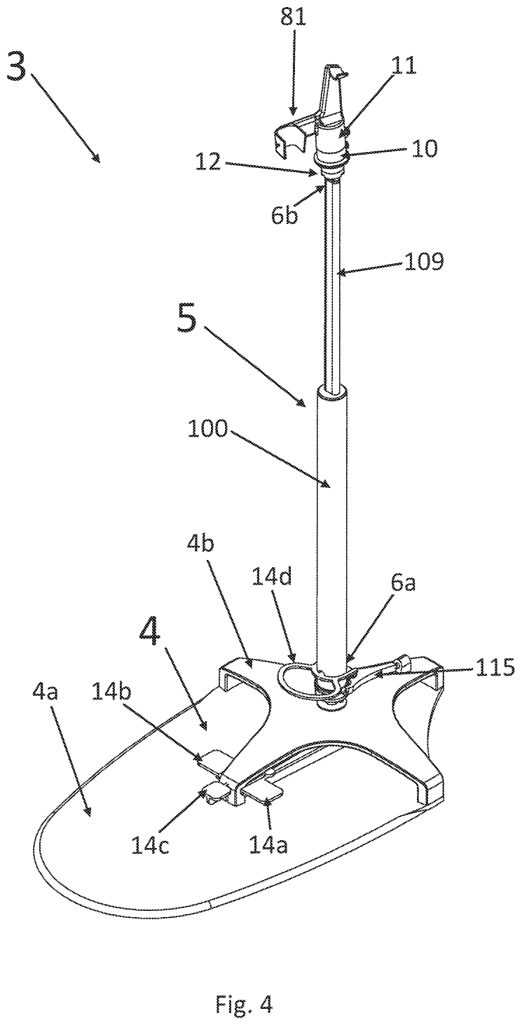

FIG. 4 shows a second embodiment of the support of the endoscope in accordance with the invention;

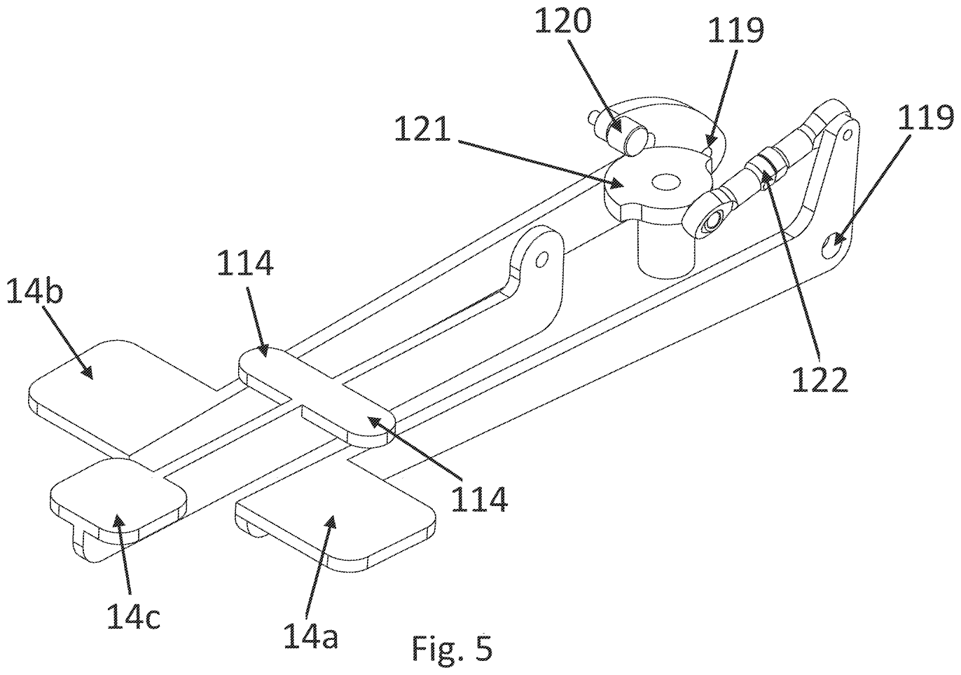

FIG. 5 shows a view of the unlocking pedals pivoted on the flat base of the support of FIG. 4;

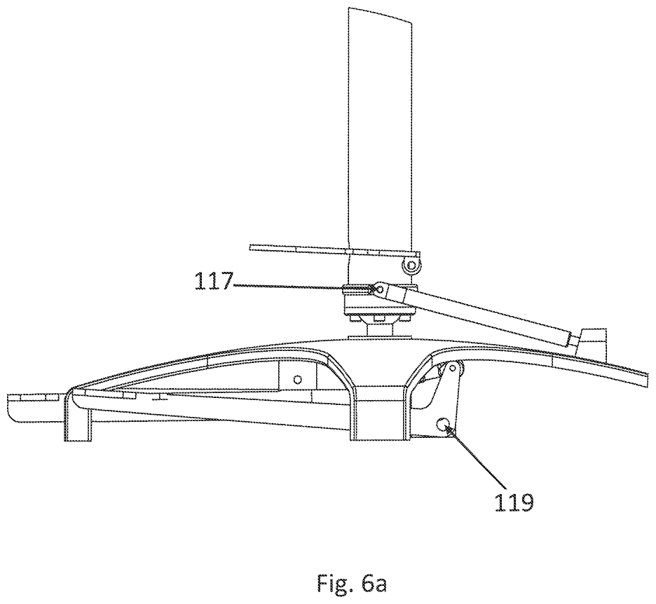

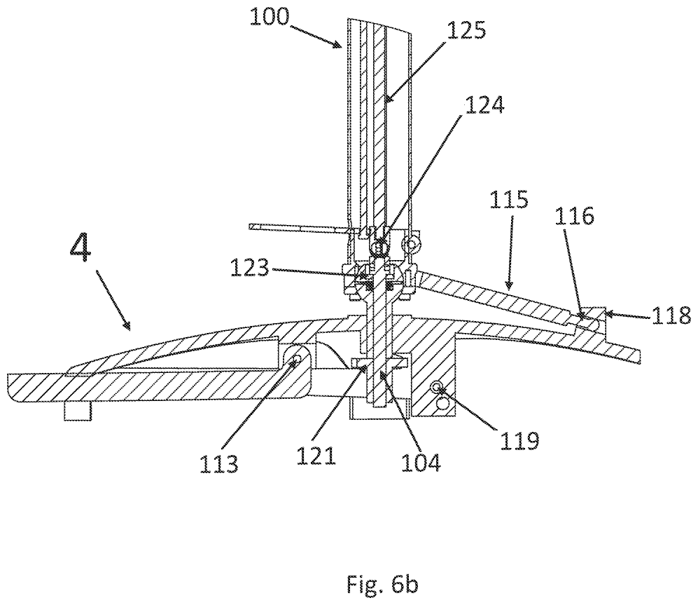

FIGS. 6a and 6b show a non-sectional view and a vertical cross-sectional view of the lower area of the support of FIG. 4 including the flat floor rest base;

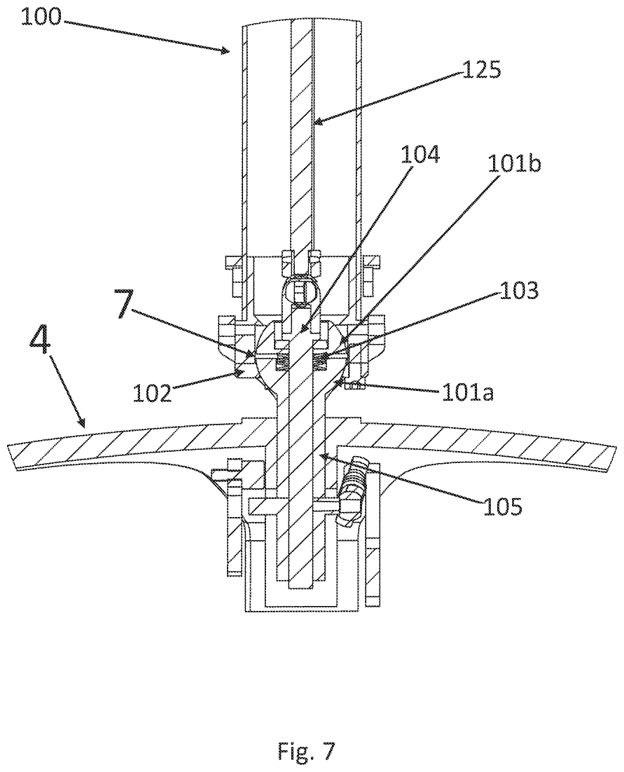

FIG. 7 shows a vertical cross section of a detail of the lower area of the support of FIG. 4 including the flat floor rest base;

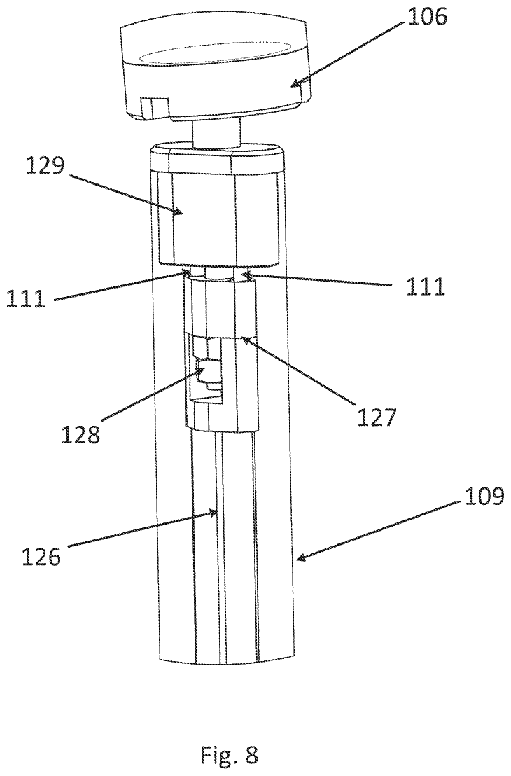

FIG. 8 shows an area of the second tube of the telescopic rod of the support of FIG. 4;

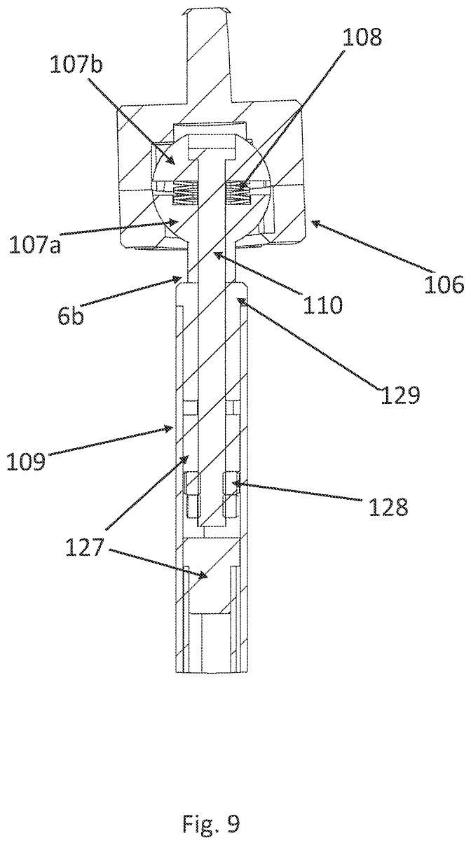

FIG. 9 shows a vertical cross section of an area of the second tube of the telescopic rod of the support of FIG. 4;

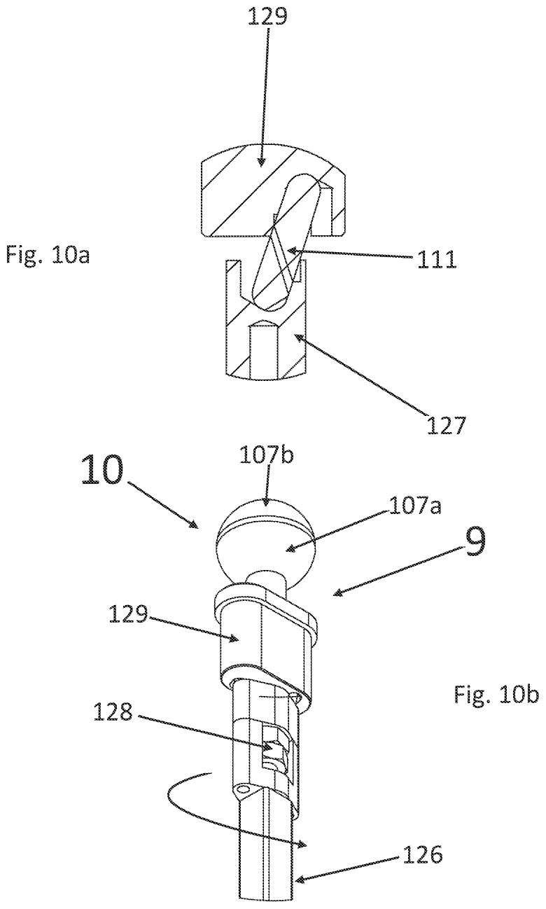

FIGS. 10a and 10b show the second mechanism for transforming a rotation of the second pedal into a linear actuation of the second tie-rod in an unlocked configuration of the degree of freedom;

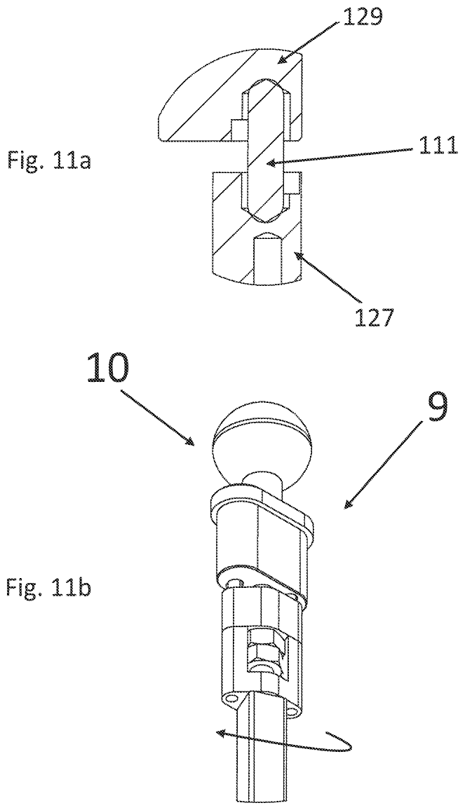

FIGS. 11a and 11b show the second mechanism for transforming a rotation of the second pedal into a linear actuation of the second tie-rod in a configuration in which the degree of freedom is blocked;

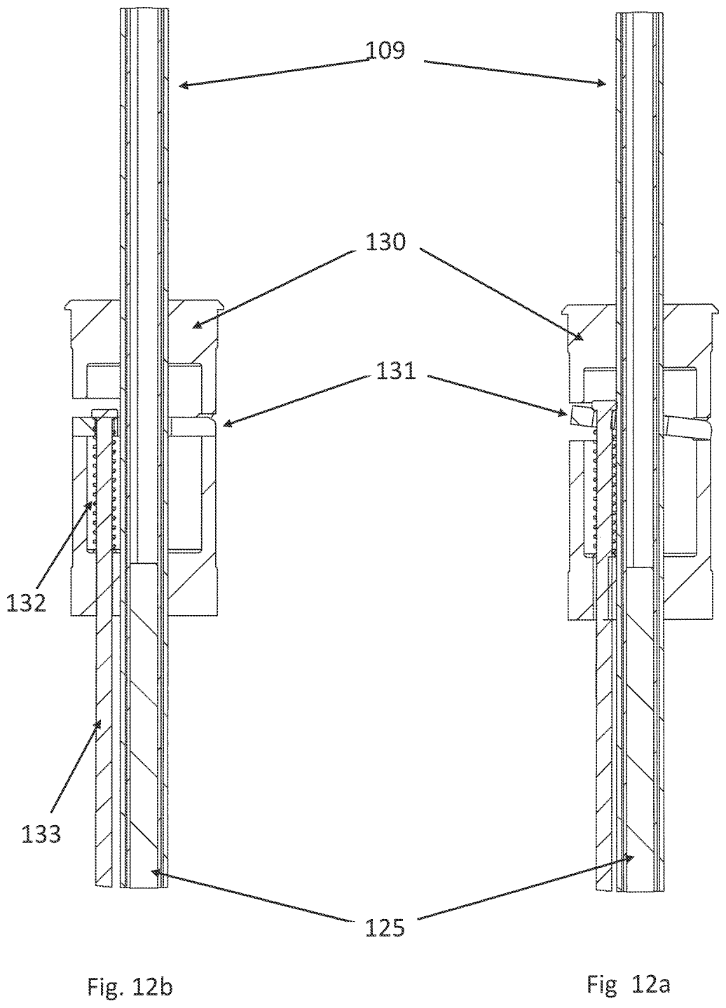

FIGS. 12a and 12b show the system for blocking the degree of translational freedom of the second tube relative to the first tube of the telescopic rod of the support of FIG. 4, in a locked and unlocked configuration;

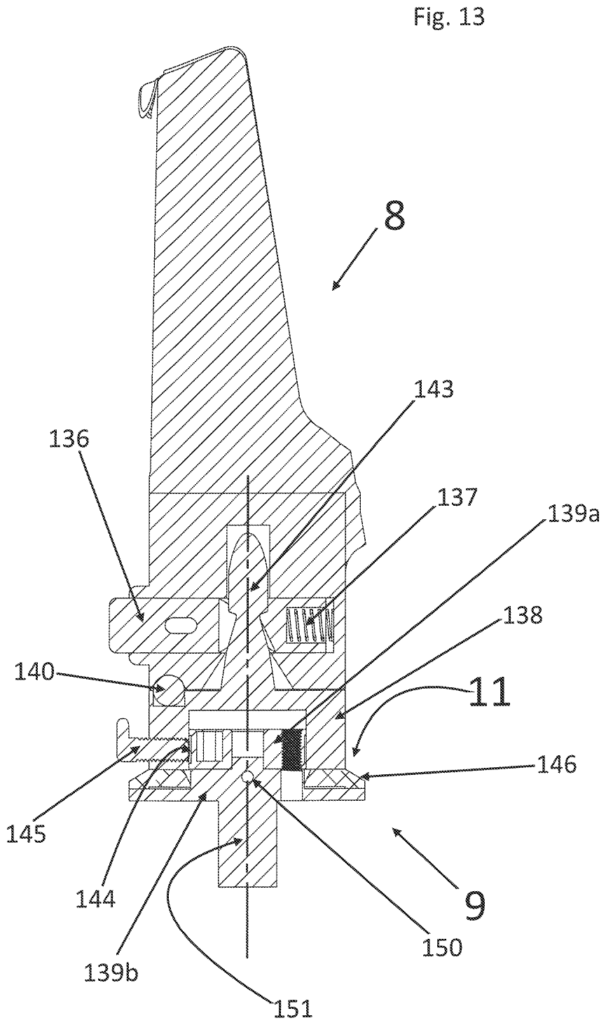

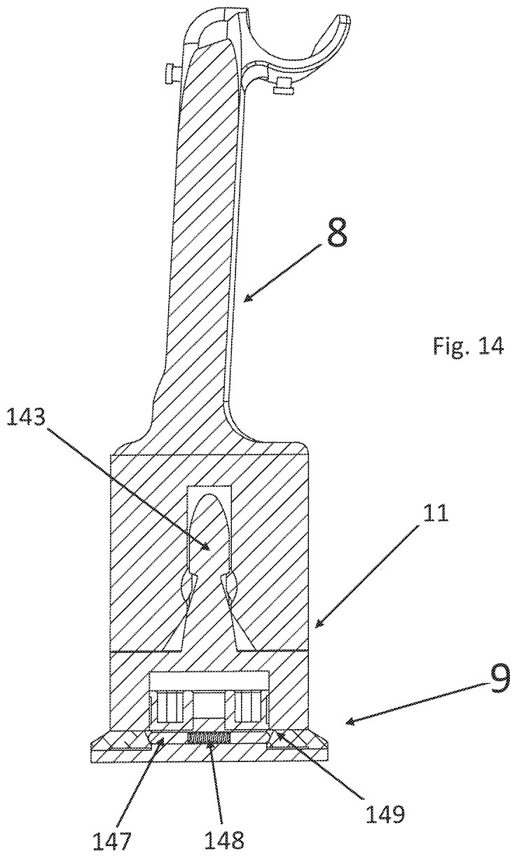

FIGS. 13 and 14 represent vertical cross sections of the connector of the endoscope of the support of FIG. 4 and FIG. 15 a component thereof;

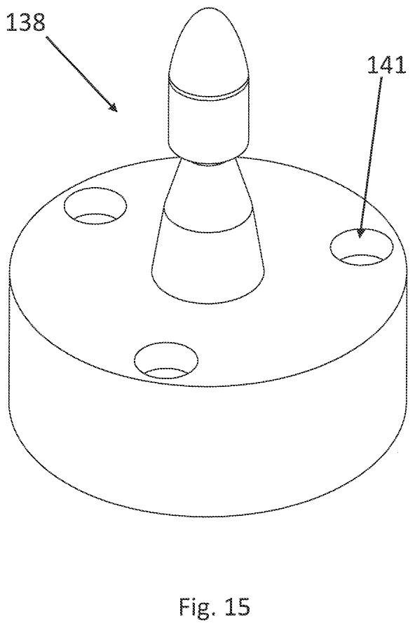

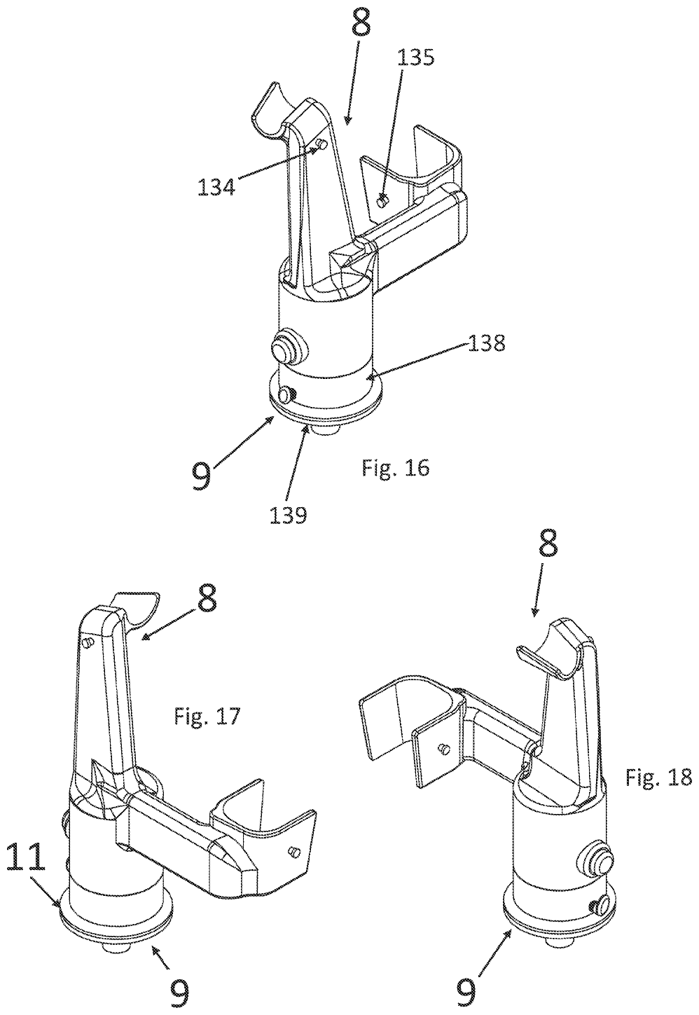

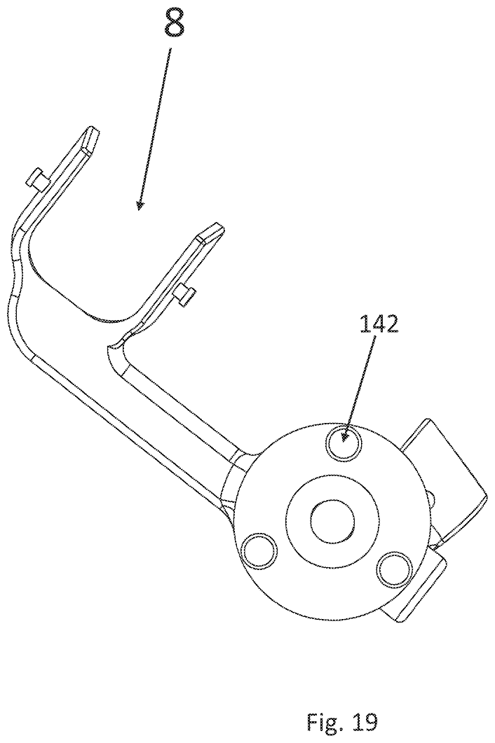

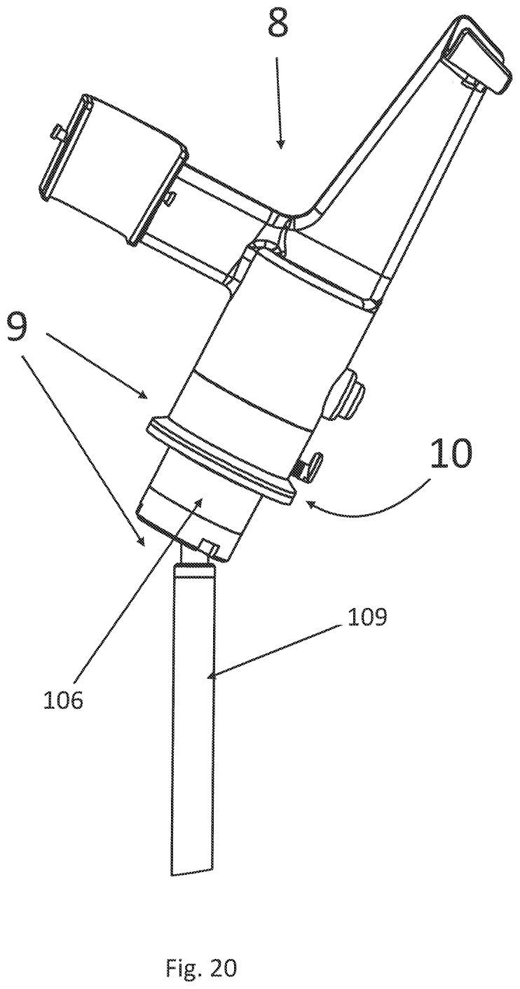

FIGS. 16 to 20 show various perspective views of the connector of the endoscope of the support of FIG. 4.

Equivalent parts in the various embodiments will be indicated with the same numerical reference.

The apparatus 1 for exploring an internal cavity of a human or animal body comprises an endoscope 2 and a support 3 for the endoscope 2.

The endoscope 2 can be any type of endoscope, rigid or flexible.

The endoscope 2 is preferably a gastroscope.

The support 3 comprises a flat floor rest base 4.

The flat rest base 4 has a large surface and heavy weight to impart positioning stability.

The flat rest base 4 is made in the form of a metal plate and has dimensions such as to enable an operator to manoeuvre the apparatus 1 while standing on the base 4.

The apparatus 1 comprises a telescopic rod 5 that extends longitudinally between a lower end 6a and an upper end 6b.

Advantageously, the telescopic rod 5 provides the support 3 with a degree of translational freedom.

The telescopic rod 5 can be any telescopic rod of a known type.

In a preferred embodiment, the rod 5 includes a first tube that comprises the lower end 6a of the telescopic rod 5 and a second tube that comprises the upper end 6b of the telescopic rod 5.

In the embodiment illustrated in FIGS. 1-3, the first and the second tube are slidable, one inside the other, and are reciprocally coupled by means of a screw system or a friction coupling system of a known type, rendering the telescopic rod 5 continuously extendable in a longitudinal direction.

In an embodiment not shown, the telescopic rod 5 can comprise a motorised movement actuator which enables extension and retraction in a longitudinal direction.

In simpler embodiments, it is possible to envisage a system of discrete regulation of the length of the telescopic rod, for example an axial series of holes provided in a length of tube in which at least one locking peg provided in the other length of tube is engageable, the locking peg being movable radially in opposition and through the action of a spring.

The telescopic rod 5 is made of metal material, preferably aluminium or titanium.

With reference once again to FIGS. 1-20, the apparatus 1 comprises a first ball joint 7 connecting the lower end 6a of the telescopic rod 5 to the rest base 4.

In particular, the ball joint 7 allows 0° to 360° rotations of the telescopic rod 5 about the longitudinal axis of extension of the rod 5, rotations of from 0° preferably up to 45° relative to the direction perpendicular to the flat rest base 4 in any plane containing the axis of the telescopic rod 5.

The ball joint 7 provides three rotational degrees of freedom to the support 3, in particular three degrees of freedom of the telescopic rod 5 relative to the flat rest base 4.

The apparatus 1 comprises an attachment connector 8, to and from which the endoscope 2 can be attached and detached, and an operative connection means 9 of the connector 8 at the upper end 6b of the telescopic rod 5.

The connector 8 is connected at one end to the operative connection means 9 and has coupling/release means at the opposite end for coupling the endoscope 2.

The operative connection means 9 provides additional degrees of freedom to the support 3, in particular degrees of freedom of the connector 8 relative to the telescopic rod 5.

The operative connection means 9 comprises an interposing block 10 between the telescopic rod 5 and the connector 8, a joint 11 connecting the block 10 to the connector 8 and a joint 12 connecting the block 10 to the upper end 6b of the telescopic rod 5.

Below we shall make reference to the embodiment of FIGS. 1-3.

The joint 11 of the operative connection means 9 is a ball joint and the joint 12 is a flat joint. The operative connection means 9 allows 0° to 360° rotations of the connector 8 about the axis of the telescopic rod 5 and rotations in planes containing the axis of the telescopic rod 5.

The interposing block 10 extends longitudinally coaxially with the telescopic rod 5 and has one end connected to the joint 12 and an opposite end connected to the ball joint 11 and housing the ball joint 11.

The ball joint 11 has a spherical cap 11a from which a pin 11b solidly joined to the connector 8 extends radially.

The ball joint 11 allows 0° to 360° rotations of the connector 8 about the axis of the block 10 and rotations in planes containing the axis of the block 10.

The block 10 has a plurality of recesses 15a, 15b at the end that houses the joint 11.

The recesses 15a, 15b are intended to receive the pin 11b when a large inclination of the connector 8 relative to the axis of the telescopic rod 5 is required.

Each recess 15a 15b of the block 10 in particular allows 0° to 90° rotations of the connector 8 connected to the ball joint 11, in a plane passing through the axis of the block 10.

The block 10 preferably comprises at least one pair of diametrically opposite recesses 15a, 15b that thus allow a −90° to 90° rotation of the connector 8.

The ball joint 11 provides three degrees of rotational freedom to the support 3, in particular three degrees of rotational freedom of the connector 8 relative to the block 10.

The joint 12 of the operative connection means 9 is a flat joint.

The flat joint 12 has a rotation axis that is coaxial with the direction of longitudinal extension of the telescopic rod 5 and comprises a rotation plate connected fixedly to the upper end 6b of the longitudinal rod 5 and rotatingly to the interposing block 10.

In particular, the flat joint 12 allows 0° to 360° rotations of the interposing block 10 relative to the axis of the telescopic rod 5.

The flat joint 12 thus provides the support 3 with a degree of rotational freedom, in particular a degree of rotational freedom of the interposing block 10 relative to the telescopic rod 5.

Advantageously, the apparatus 1 comprises unlocking means 13a, 13b, 13c for unlocking the degrees of freedom of movement of the support 3 for the endoscope 2.

In particular, the apparatus 1 comprises a first unlocking means 13a for unlocking the degrees of freedom of the support 2 which comprises a locking pedal 14 actuable so as to lock and unlock the ball joint 7 connecting the lower end 6a of the telescopic rod 5 to the flat rest base 4.

The first unlocking means 13a comprises a locking plate 16 hinged to an anchorage plate 17 fixed to the rest base 4.

The locking plate 16 is movable in opposition and through the action of an elastic element 18 which connects it to the anchorage plate 17.

Interposed between the anchorage plate 17 and the locking plate 16 is the ball joint 7, which projects from a through opening of the locking plate 16.

The elastic element 18 is for example a helical spring tensioned by traction which maintains the locking plate 16 normally engaged against the joint 7 in a locked position.

The pedal 14 is pivoted on the anchorage plate 17 and comprises a first arm 19 for picking up movement in order to receive a command from an operator's foot and an arm 20 for pushing against the locking plate 16.

When the first arm 19 for picking up the movement of the pedal is pressed, the second arm 20 for pushing rotates around the pivot and acts against the locking plate 16 and, by deforming the elastic element 18, disengages the locking plate 16 from the position of locking the ball joint 7, thus freeing the ball joint 7.

The apparatus 1 comprises a second unlocking means 13b for unlocking the degrees of freedom of the operative connection means 9.

In particular, said second unlocking means 3b comprises a first knob 21, which actuates a braking element to gradually block the rotation of the joint 11.

The second unlocking means 13b comprises a second knob 22, which actuates a locking pin to block the rotation of the joint 12 of the operative connection means 9.

The apparatus 1 comprises a third unlocking means 13c for unlocking the degrees of freedom of the telescopic rod 5 which comprises a knob, not shown in the figure, which actuates a pin or a ring for blocking the extension of the telescopic rod 5.

Advantageously, the operative connection means 9 and/or the ball joint 7 are manually actuable.

In a different embodiment of the invention, the operative connection means 9 and/or the ball joint 7 are motorised.

Advantageously, the motorised operative connection means 9 and the ball joint 7 are remotely manoeuvrable.

Advantageously, the support 3 comprises at least a graduated scale for indicating the deviation of the operative connection means 9 from a reference position.

In particular, the joint 12 of the operative connection means 9 is provided with a graduated scale which is integrated in said rotating plate and resettable so as to keep a reference of the deviation from a pre-set reference position.

Advantageously, the apparatus 1 comprises at least a graduated scale for indicating the deviation of the ball joint 7 connecting the lower end 6a of the telescopic rod 5 to the rest base 4 from a reference position.

Advantageously, the connector 8 and the endoscope 2 are provided with a mutual quick coupling means.

Below we shall make reference to the embodiments of FIGS. 4-20.

In this case the joint 11 connecting the block 10 to the connector 8 is a flat joint and the joint 12 connecting the block 10 to the upper end 6b of the telescopic rod 5 is a ball joint.

The unlocking means comprise pedals 14a, 14b, 14c, 14d.

The flat floor rest base 4 comprises a flat plate 4a and a bracket 4b fixed onto the flat plate 4a.

The pedals 14a, 14b, 14c are pivoted on the bracket 4b of the flat base 4 whilst the pedal 14d is pivoted on the telescopic rod 5, in particular on the first tube 100 which comprises the lower end 6a of the telescopic rod 5.

In this case, as we shall see in detail below, the connector 8 and the interposing block 10 are provided with a mutual quick coupling and release means.

The first ball joint 7 comprises a first ball positioned in a first housing 102 provided in the first tube 100 of the telescopic rod 5.

The first ball comprises a first half-ball 101a and a second half-ball 101b actuable towards the first half-ball 101a, in opposition to at least a first spring 103, by a first pedal 14b.

The unlocking means comprise a first mechanism for transforming a rotation of the first pedal 14b into an actuation of a first tie-rod 104 for pulling the second half-ball 101b towards the first half-ball 101a.

The first half-ball 101a extends with a vertical pin 105 for fixing to the flat base 4, in particular to the bracket 4b.

The fixing pin 105 is tubular and axially and slidingly receives the first tie-rod 14.

The joint 12 connecting the block 10 to the upper end 6b of the telescopic rod 5 is a second ball joint comprising a second ball positioned in a second housing 106.

The second ball comprises a first half-ball 107a and a second half-ball 107b actuable towards the first half-ball 107a, in opposition to a second spring 108, by a second pedal 14a.

The first half-ball 107a is fixed to the second tube 109 of the telescopic rod 5.

The unlocking means comprise a second mechanism for transforming a rotation of the second pedal 14a into a linear actuation of a second tie-rod 110 for pulling the second half-ball 107b towards the first half-ball 107a.

The second tie-rod 110 is solidly joined in rotation but not in translation to the first tie-rod 105.

The second mechanism comprises inclinable spacer pins 111 for transforming a rotation of the first tie-rod 105 into a rototranslation of the second tie-rod 110.

The solution shown in FIGS. 4-20 is described in even greater detail below.

The support 3 is composed, starting from the bottom, of the base 4, which is conformed in such a way that the operator stands on top of it, and which has a bracket 4b that in turn supports the first lower ball joint, the actuator pedals 14a and 14b, and the central pedal 14c, pivoted in 113 to the bracket 4b, which has the function of lowering both of the pedals 14a and 14b by pushing them with the tabs 114.

Moving up the support 3 we find the pair of telescopic tubes 100, 109, the second upper ball joint and, finally, the section with a flat joint between the connector 8 and the block 10.

Hereinafter, “stably” or “normally” locked will be understood to mean a joint, or a state of the joint itself, that remains braked without the operator's intervention; “stably” or “normally” unlocked will be understood to mean a joint, or a state thereof, which remains free without the operator's intervention.

The plate 4a of the base 4 is large and extends towards the operator in such a way that the latter is positioned on top of it during the procedure. This conformation has been designed with the aim of ensuring the maximum stability of the whole apparatus, thanks to the large rest surface and the weight of the operator. The plate 4a can also be separate from the rest of the system, so as to facilitate its transport or use in an independent manner in settings in which a frequent moving of the support 3 proves convenient.

The bracket 4b supports the anti-rotation fork 115, which constrains the rotation of the first tube 100 by means of terminal joints 116, 117, in which the joint 117 connecting to the first tube 100 comprises bearings mounted on an axis perpendicular to the axis of the first tube 100 and sliding along an outer circumference of the first tube 100 and the joint 116 connecting to the bracket 4b comprises a shank of the fork 115 slidingly engaged in a slot 118.

The first lower ball joint has the two half-balls 101a, 101b in a configuration that is normally expanded by the first springs 103, which press them against the inner surfaces of the housing 102.

Braking is ensured by the friction of the contact surfaces and the force exerted by the first springs 103.

In order to free the joint from the braking effect and thus be able to move the tube 100 and everything connected to it in space, one acts by pressing the pedal 14b, with the pivot in 119, which, with the roller 120, pushes on a plate 121, in turn connected to the first tie-rod 104, which pulls the second half-ball 101b downward.

For the upper ball joint, stably locked, braking thus takes place thanks to the force of the springs 108, which act by expanding the two half-balls 107a, 107b, which push on the inner surfaces of the housing 106.

The unlocking system takes place through the traction of the second tie-rod 110, which, by pulling the second half-ball 107b downwards, lightens the pressure on the inner surface of the housing 106.

The method whereby the second tie-rod 110 is pulled, however, must take account of the fact that the pedal 14a which controls it is also positioned on the plate 4b, likewise pivoted on the axis 119, and of the fact that the telescopic connection between the first tube 100 and the second tube 109 of the telescopic rod 5 is interposed between the second ball joint and the pedal 14a.

In order to obtain the action of traction, therefore, a system has been implemented which transforms the linear movement of a connecting rod 122 moved by the pedal 14a and connected to the plate 121 into a rotational movement.

This rotation progressively involves the first tie-rod 104, which is free to move in the absence of friction thanks to the thrust bearing 123, a cardan joint 124 connected to the first tie-rod 104, a first rod 125 connected by means of the cardan joint 124 to the first tie-rod 104, a second rod 126 telescopically connected to the first rod 125, with which it is solidly joined in rotation, and a terminal 127 of the second rod 126.

The elements 124, 125, 126 and 127 are all contained in the pair of telescopic tubes 100, 109. The sections 125 and 126 remain free to slide linearly relative to each other, but as they have a non-circular cross section they integrally transmit the rotation.

The cardan joint 124 ensures that the rotational motion is effectively transmitted without affecting or being affected by the angles of inclination it is possible to reach thanks to the first ball joint.

The element 127 is free to slide linearly inside the tube 109.

At this point the rotational motion must be transformed again into linear; this operation is achieved by means of the pins 111, which, on being brought from the inclined rest position to the vertical one, increase the distance between the element 127, solidly joined to the second tie-rod 110 thanks to the nuts 128, and the element 129, solidly joined to the second tube 109 of the telescopic rod 5 and to the first half-ball 107a of the second ball joint.

The second tube 109 resists the force applied and transmitted by the elements 104, 124, 125, 126 and 127 thanks to its non-circular cross section, which constrains its rotation relative to the element 130, in turn solidly joined to the first tube 100.

The telescopic joint of the rod 5 comprises the element 130, formed by a casing stably constrained to the first tube 100 and passed through by the second tube 109.

A braking element 131, pivoted in the cavity of the casing, acts directly on the body of the second tube 109, which passes through it and is pushed by a spring 132, which ensures that the braking element 131 is inclined and engages the second tube 109, braking it thanks to the resulting interference.

The action of unlocking the brake takes place through the traction that is exerted by a tie-rod 133 moved by the pedal 14d on the braking element 131, thereby opposing the force of the spring 132, reducing the inclination of the braking element 131 and lightening the pressure on the second tube 109, which is thus freed from constraint.

The connector 8 is created specifically to ensure a solid connection with the endoscope, thanks to the two arms supporting the instrument at the level of the handpiece and of the tube which comes out of the rear part of the latter.

The endoscope is supported by virtue of its shape, and rendered solidly joined with elastic bands secured to the pegs 134, 135.

The connector 8, in its lower part, acts as a female element for the quick release system, and has a pushbutton 136 and a spring 137 adapted to this purpose inserted into it.

The interposing block 10 comprises a first upper component 138 solidly joined in the direction of an axis 152 to a second lower element 139a, 139b of the interposing block 10.

However, the first upper component 138 can rotate about the axis 152 relative to the second lower element 139a, 139b, which is in turn fixed to the housing 106 of the two half-balls 107a, 107b of the second ball joint.

The elements which enable the quick release are the bottom of the connector 8, the pushbutton 136 coupled to the spring 137, the first component 138 of the interposing block 10, the second element 139a, 139b of the interposing block 10 and balls 140, set in seats 141 present on the first component 138 and self-centring in seats 142 present on the bottom of the connector 8.

When the pushbutton 136 is pressed by the operator, the same slips out from the undercut geometry of a pin 143 of the first component 138 of the interposing block 10 and the connector 8 is freed.

The coupling step, by contrast, can be performed without exerting any pressure on the pushbutton 136 but simply by exerting pressure downwards on the connector 8; the balls 140, fixed in the seats 141, will be inserted into the seats 142, thereby constraining the relative rotation between the two components.

The flat joint 11 is defined between the two components 138, and 139a, 139b of the interposing block 10.

The rotation of the assembly formed by the connector 8 and the first component 138 of the interconnecting block 10 takes place thanks to the possibility that the latter has of rotating about its axis 152 on the second component 139a, 139b of the interconnecting block 10.

The upper piece 139a of the second component 139a, 139b of the interconnecting block 10 has a groove 144 on which a pin 145 acts with the objective of totally or partially blocking the movement allowed by the flat joint 11.

The lower piece 139b of the second component 139a, 139b of the interconnecting block 10 instead has the task of firmly fixing the whole assembly 138, 139a, 139b to the housing 106 of the two half-balls 107a, 107b of the second ball joint 12 as well as of acting as a support for a vernier 146 that is present to give a resettable reference to the operator.

The vernier 146 is rotatable on an annular surface of the second component 139a, 139b of the interconnecting block 10 and is kept in place by pins 147 pressed by a spring 148 against an inclined inner surface 149 of the vernier 146.

The pins 147 are seated in a hole 150 that passes through the second component 139a, 139b of the interconnecting block 10.

The function performed by the flat joint 11 is not so much that of adding degrees of freedom to the system (rotation is already made possible by unlocking the second ball joint), but more than anything else the possibility, once the whole system is locked, of acting on fine adjustments and thus freeing the movement (rotation) that most influences the position of the terminal part of the endoscope inside the patient.

The presence of the vernier 146 is fundamental for creating the references which permit the replicability of movements in some procedures, in which the alignment of the operator channel used relative to the surface of tissues is crucial for the success of the procedure.

Finally, the possibility of quickly releasing the connector 8 must be understood almost as an “emergency button”: the system is designed to support the endoscope throughout the entire procedure, from its entry into the patient until its exit from the latter; in some cases, however, it could be necessary for the operator to remove the instrument rapidly in order to replace it with another, or else to end a freehand exploration of the cavity.

The peculiar characteristic of the apparatus lies in the number of degrees of freedom, which when unblocked do not limit the movements of the operator at all.

Moreover another peculiar characteristic of the apparatus lies in the characteristic of being able to unlock or block the joints, and therefore these degrees of freedom, independently of one another.

The apparatus 1 for exploring an internal cavity of a human or animal body thus conceived is susceptible of numerous modifications and variants, all falling within the scope of the inventive concept; moreover, all of the details can be replaced with technically equivalent elements. The materials used, as well as the dimensions, may in practice be any whatsoever, according to needs and the state of the art.

1. An apparatus (1) for exploring an internal cavity of a human or animal body, comprising an endoscope (2) and a support (3) for the endoscope (2), characterised in that said support (3) comprises a flat floor rest base (4), a telescopic rod (5) having a lower end (6a) and an upper end (6b), a first ball joint (7) connecting said lower end (6b) of the telescopic rod (5) to said floor rest base (4), a disconnectable attachment connector (8) to said endoscope (2), and an operative connection means (9) connecting said connector (8) to said upper end (6b) of said telescopic rod (5), said operative connection means (9) comprising an interposing block (10) between said connector (8) and said telescopic rod (5), a joint (11) connecting said block (10) to said connector (8) and a joint (12) connecting said block (10) to said upper end (6b) of said telescopic rod (5).

2. The apparatus (1) for exploring an internal cavity of a human or animal body according to claim 1, characterised in that said joint (11) connecting said block (10) to said connector (8) is a ball or respectively flat joint and said joint (12) connecting said block (10) to said upper end (6b) of said telescopic rod (5) is a flat or respectively ball joint.

3. The apparatus (1) for exploring an internal cavity of a human or animal body according to claim 1, characterised in that it has unlocking means for unlocking the degrees of freedom of said support (3).

4. The apparatus (1) for exploring an internal cavity of a human or animal body according to claim 1, characterised in that said unlocking means comprise pedals (14, 14a 14b, 14c, 14d).

5. The apparatus (1) for exploring an internal cavity of a human or animal body according to claim 1, characterised in that at least one of said pedals (14a, 14b, 14c) is pivoted on said telescopic rod (5).

6. The apparatus (1) for exploring an internal cavity of a human or animal body according to claim 1, characterised in that said support (3) comprises at least a graduated scale for indicating the deviation of said operative connection means (9) from a reference position.

7. The apparatus (1) for exploring an internal cavity of a human or animal body according to claim 1, characterised in that said connector (8) and said endoscope (2) are provided with a mutual quick coupling and release means.

8. The apparatus (1) for exploring an internal cavity of a human or animal body according to claim 1, characterised in that said connector (8) and said interposing block (10) are provided with a mutual quick coupling and release means.

9. The apparatus (1) for exploring an internal cavity of a human or animal body according to claim 4, characterised in that said first ball joint comprises a first ball positioned in a first housing, said first ball comprising a first half-ball (101a) and a second half-ball (101b) which is actuable towards the first half-ball (101a) in opposition to at least a first spring (103) by a first of said pedals (14b) pivoted on said flat base (4).

10. The apparatus (1) for exploring an internal cavity of a human or animal body according to claim 9, characterised in that said unlocking means comprise a first mechanism for transforming a rotation of the first pedal (146) into a linear actuation of a first tie-rod (104) for pulling said second half-ball (101b) towards said first half-ball (101a).

11. The apparatus (1) for exploring an internal cavity of a human or animal body according to claim 10, characterised in that said telescopic rod (5) comprises a first tube (100) having said lower end (6a) and a second tube (109) having said upper end (6b) and which is slidable along said first tube (100) so as to adjust the distance between said upper end (6b) and said lower end (6a), said first half-ball (101a) extending with a vertical pin (105) for fixing to said flat base (4).

12. The apparatus (1) for exploring an internal cavity of a human or animal body according to claim 11, characterised in that said joint (12) connecting said block (10) to said upper end (6b) of said telescopic rod (5) is a second ball joint comprising a second ball positioned in a second housing (106), said second ball comprising a first half-ball (107a) and a second half-ball (107b) which is actuable towards the first half-ball (107a) in opposition to at least a second spring (108) by a second pedal (14a) pivoted on said flat base (4), said unlocking means comprising a second mechanism for transforming a rotation of the second pedal (14a) into a linear actuation of a second tie-rod (104) for pulling said second half-ball (107b) of said ball towards said first half-ball (107a) of said second ball.

13. The apparatus (1) for exploring an internal cavity of a human or animal body according to claim 12, characterised in that said first half-ball (107a) of said second ball is fixed to said second tube (109), said second tie-rod is solidly joined in rotation but not in translation to said first tie-rod (104), and said second mechanism comprises inclinable spacer pins (111) for transforming a rotation of the first tie-rod (104) into a rototranslation of the second tie-rod (110).
